(19) United States
(12) Patent Application Publication
(10) Pub. No.: US 2010/0305680 A1
(43) Pub. Date: Dec. 2, 2010

(54) DEVICE FOR PERCUTANEOUS VASCULAR INTERVENTION

(75) Inventor: Hansgeorg Schaaf, Reichertshausen (DE)

Correspondence Address: MICHAEL BEST & FRIEDRICH LLP, 100 E WISCONSIN AVENUE, Suite 3300, MILWAUKEE, WI, 53202, US

(73) Assignee: POLYDIAGNOST GMBH, Pfaffenhofen (DE)

(21) Appl. No.: 12/747,184

(22) PCT Filed: Dec. 10, 2008
(86) PCT No.: PCT/DE2008/002081

§ 371 Date: Aug. 12, 2010

(30) Foreign Application Priority Data
Dec. 10, 2007 (DE) 20 2007 017 233.8

(51) Int. Cl. A61F 2/84

(52) U.S. Cl. 623/1.11

(57) ABSTRACT

A device for percutaneous vascular intervention, particularly for performing a stent implantation, having a guide catheter 1 and a guide wire that is guided in a catheter lumen of the guide catheter 1 to the intervention location in the vessel, wherein two control wires 2, 3 connected to the distal catheter end 4 are provided for controlling the distal catheter end 4.

### BACKGROUND OF THE INVENTION

The invention relates to a device for percutaneous vascular intervention, particularly for performing a stent implantation, having a guide catheter and a guide wire that is guided in a catheter lumen of the guide catheter to the intervention location in the vessel.

The percutaneous vascular intervention is a non-surgical catheter-invasive measure that is frequently used for opening stenosed vessels. The catheter system that has been used for this purpose so far consists substantially of three parts, namely of a guide catheter, a guide wire, and a probe. In this measure, the guide wire is first of all pushed forward in a catheter lumen of the guide catheter up to the intervention location in the vessel, for instance, up to a stenosis, and serves to support the probe, wherein the probe is pushed forward to the stenosis via the guide wire. By means of the probe, the therapeutic mechanism to be used, for instance, a balloon, is taken to the intervention location. By means of the balloon, the vessel is expanded, for instance, in the region of the stenosis. It is further possible to push forward a balloon catheter stent system via the guide wire up to the stenosis region and to implant the stent by expanding the balloon. This intervention is relatively complex to perform. A balloon stent catheter system is, for instance, known from EP 0 779 062 A1.

The use of guide catheters with a small diameter, for instance, 5 Fr and less, is more efficient with percutaneous coronary interventions than the use of catheters with a larger diameter. It is especially when performing the intervention via arteria femoralis that a reduced number of peripheral, vascular complications results. Furthermore, a reduction of the duration of the entire procedure and of the contrast medium usage is achieved. Difficulties arise during the controlled guiding of the guide catheter. So far it has therefore not been possible to find an access to vessels that are branched off in vascular arcades as is, for instance, the case with the arteria carotis.

### SUMMARY OF THE INVENTION

It is an object of the invention to provide a device of the initially mentioned type, wherein the percutaneous, particularly coronary intervention is also possible in vessels that are branched off from a vascular arcade.

In the invention, the guide catheter is provided with two control wires. They are guided to be moved longitudinally in the longitudinal direction of the guide catheter and are firmly connected to the distal catheter end. This may be performed by gluing or by force fit, as is, for instance, known from WO 2005/074787 A1. On actuation of the first control wire by means of a slider connected with the distal end of the control wire the distal catheter end can be bent in a first direction. On actuation of the second control wire, also by means of an allocated slider that is connected with the proximal end of the control wire, the distal end of the guide catheter can be bent in a second direction opposite to the first direction. The two bendings of the distal end of the guide catheter are preferably performed in the same plane. On bending by means of the second control wire, the first control wire is preferably fixed against a longitudinal displacement with the catheter end bent in the first direction. This makes it possible to guide a directed movement of the distal catheter end in a controlled manner in a vessel branched off from the vascular arcade.

This renders it possible, for instance, to head for the arch of the aorta with a bending of the distal catheter end which has a relatively large radius, and to subsequently move the distal catheter end into the arteria carotis with a bending of narrow radius (distance from the distal catheter end) of approx. 15 mm in the direction opposite to the first bending.

The two control wires are positioned in the catheter cross-section of the guide catheter preferably diametrically to each other with respect to the longitudinal axis of the catheter. Preferably, each of the two control wires is designed to be free from twist effects around the respective longitudinal axis thereof. This means that a rotary motion applied at the proximal end of the control wire is transferred to the distal wire end with an equal angle of rotation. Due to this property of the two control wires, the guide catheter is also designed to be free from twist effects along the entire length about the longitudinal axis thereof. A rotary motion applied to its proximal end is thus transferred to the distal catheter end with an equal angle of rotation.

It is in particular with an intervention via arteria femoralis that a controlled guide catheter is used which comprises two multi-lumen catheter tubes of different rigidity. A first catheter tube consists of a relatively rigid material that has, however, still sufficient elasticity for being pushed forward in the vessel. A second catheter tube that is adapted to be bent retrogradely by means of the control wires is connected with the distal end of the first catheter tube. The control wires are connected with the distal end of the second catheter tube for the controlled bending of the catheter tube and are guided to be moved longitudinally in two assigned catheter lumens.

By the controlling of the second catheter tube that is adapted to be bent retrogradely if required, an adaptation to the vessel shape on advancing of the catheter in the vessel is achieved. Above all, peripheral vascular complications are avoided by that.

The control wires and the guide catheter are adapted to be twisted against each other about the longitudinal axis of the catheter, wherein it is possible to steplessly fix different positions of the angle of rotation. To this end, the proximal end of the first catheter tube of the guide catheter may be connected with an adapter, and the proximal ends of the control wires may be connected with dedicated sliders. By the twisting of the adapter and of the respective slider against each other it is possible to adjust the different positions of the angles of rotation of the guide catheter and of the respective control wire and to make them visible to the surgeon. This also renders it possible to adjust the respectively desired direction of bending of the distal catheter end. Due to the torsional rigidity of the respective control wire the bent distal catheter end follows the position of the angle of rotation of the control wire with respect to the catheter.

At the distal catheter end, a balloon may be provided which is, via a corresponding lumen in the guide catheter, filled with a filler, for instance, saline solution or a mixture of contrast medium and saline solution, and which may be expanded in this process. This balloon proves to be advantageous in particular in the case of acute bends or curves in the tissue. By the expanding of the balloon the vascular wall is stiffened in the region of the bending, so that, when the guide wire is pushed out beyond the distal end of the guide catheter, the guide wire can be pushed further without vascular complications. By means of the expanded balloon, the guide catheter is blocked in the vessel while the guide wire is pushed beyond the distal catheter end. After the emptying of the balloon the guide catheter may be pushed further, following the guide wire. This stepwise moving of the guide catheter is used in particular in vessel regions with acute bends or curves.

The pushing forward of the guide catheter is performed in a conventional manner under radiographic control; to this end, appropriate markers, for instance, metal markers, may be incorporated at the distal end of the guide catheter. Instead of the radiographic control, ultrasonic control may also be performed.

The guide catheter further comprises a working lumen in which the guide wire may be guided. Furthermore, the diameter of the working lumen may be dimensioned such that a probe with a therapeutical mechanism, for instance, with a balloon, or a balloon stent catheter, may be guided up to the intervention location, for instance, into the region of a stenosis. Instead of a probe, a push rod may also be moved through the working lumen, wherein a stent is taken up to the region of the stenosis by means of the push rod. This may, for instance, be a stent that expands automatically and that expands after leaving the distal end of the working lumen. Such stents, for instance, of nitinol are known.

It is further possible to take a device for micro probe surgery, for instance, a micro drill, to the intervention location through the working lumen, so as to perform a rotablation e.g. in the case of strongly fibrotic, calcified or sidled stenoses. It is also possible to produce passages at the stent to vascular branches covered by the stent by means of the micro drill at the distal end of the second catheter tube. Appropriate devices for performing micro surgery, in particular drilling tools, which are adapted to be pushed through the working lumen of the guide catheter are described in DE 197 44 856 A1 and in DE 199 56 517 A1.

### BRIEF DESCRIPTION OF THE DRAWINGS

By means of the Figures, the invention will be explained in more detail with an embodiment.

There show:

FIG. 1a first embodiment; and

FIG. 2a cross-sectional illustration through a guide catheter that is used in the embodiment of FIG. 1.

### DETAILED DESCRIPTION

The illustrated embodiment comprises a guide catheter 1 that may comprise two multi-lumen catheter tubes of different rigidity. It is, however, also possible to use a catheter tube of uniform rigidity in the longitudinal extension. A first catheter tube 7 consists of a relatively rigid material. The first catheter tube 7 is, however, capable of being bent to such an extent that it adapts itself to vessel progressions that deviate slightly from a rectilinear extension.

The proximal end of the first catheter tube 7 is connected with an adapter 9 of a rigid material. Various connections open into the adapter 9, said connections, as will still be explained, providing access to a working lumen 11, a balloon lumen 14, and possibly to a further lumen 15.

The distal end of the first catheter tube 7 is connected with a second catheter tube 8. The second catheter tube 8 is designed to be so flexible that it is adapted to be bent retrogradely. This bending is controlled by two control wires 2 and 3. The control wires 2 and 3 are guided to be moved in the longitudinal direction in allocated catheter lumens 5 and 6 and are connected at their distal ends with the distal end of the second catheter tube 8. At the proximal ends, the control wires 2 and 3 are guided through the adapter 9 and two guide tubes 16, 17 that are firmly connected with the adapter 9. The proximal ends of the control wires 2 and 3 are firmly connected with allocated sliders 12, 13, for instance, by means of locking screws 22, 23. The sliders 12, 13 are, for instance, disposed in longitudinal slits to be guided in the longitudinal direction in a slider housing or in separate slider housings 18, 19. The sliders 12, 13 may be connected in a known manner with hand grips, in particular finger grips, that are not illustrated in detail.

The slider housings 18, 19 are adapted to be connected in a rotation resistant manner with the guide tubes 16, 17 by means of locking screws 20, 21, and via the adapter 9 in a rotation resistant manner with the first catheter tube of the guide catheter 1. By releasing the respective locking screw 20 or 21 it is possible to rotate the allocated control wire 2 or 3 about the longitudinal axis thereof and to thus adjust an additional deflection of the distal catheter end 4 about the catheter tube axis. The stepless twist of the respective control wire 2 or 3 and the respective position of the angle of rotation may be fixed by means of the respective locking screw 20 or 21. Instead of the locking screws 20-23, other suitable fixing means may also be used. From the position of the respective slider housing 18 or 19 vis-à-vis the adapter 9 it is possible to visually examine the deflection of the distal catheter end 4 with respect to the catheter tube caused by the twist of the control wire.

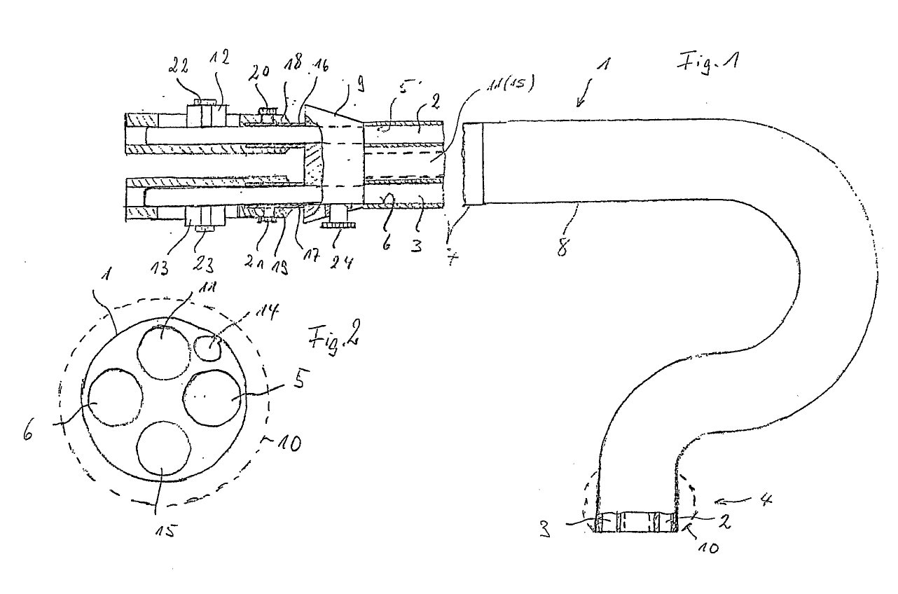

As is illustrated in FIG. 1, a first bending of the distal catheter end may, for instance, be moved by means of the control wire 3. In FIG. 1, this first bending of the catheter tube is performed with respect to the longitudinal axis to the right. By means of the control wire 2 it is possible, by maintaining the first bending, to bend the distal catheter end 4 with a second bending of a preferably smaller radius. The second bending is preferably performed in the same plane as the first bending. As explained above, it is, however, also possible to select a desired direction for the respective bending by twisting the respective control wire 2 or 3.

In order to maintain the first bending, a locking means, for instance, in the form of a locking screw 24 may be provided which fixes the initially actuated control wire 3 with respect to the adapter 9 and thus with respect to the guide catheter. This bending is also maintained during the control of the second bending.

Between the slider 13 and the slider housing 19 a self-locking generated by static friction, clamping, or otherwise may also be effective, so that the adjusted first bending is maintained even if the slider 13 is released. To maintain also the second bending, a self-locking may be provided between the slider 12 and the slider housing 18. Furthermore, an additional locking screw or an equivalent means may be provided for holding the control wire 2.

A guide wire may be moved by a corresponding connection at the adapter 9 and a working lumen 11 in the guide catheter 1 up to the distal catheter end and beyond this distal end. As already explained above, this guide wire is in particular useful if the guide catheter 1 and in particular the second catheter tube 8 have to be guided in acute bends or curves of a vessel. In this case, as already explained, the portion of the guide catheter 1 which has already been introduced in the vessel is blocked by means of a balloon 10 provided at the distal catheter end. This happens in that the balloon 10 is inflated by means of a filler, for instance, saline solution or a mixture of saline solution and contrast medium, as is illustrated with dashed lines in FIG. 1. This causes an additional stiffening of the vessel in the region of the bending thereof, and the guide wire is adapted to be pushed further beyond the distal end of the bent second catheter tube without impairing the vascular wall. The filler is supplied via a balloon lumen 14 in the guide catheter 1. In the balloon lumen 14, openings for filling and emptying the balloon are provided in the region of the balloon 10. After the emptying of the balloon 10 the guide catheter 1 may, supported by the guide wire, also be pushed further. These steps may be repeated during the pushing forward of the guide catheter 1 and the guide wire.

As soon as the guide catheter 1 has reached with its distal catheter end 4 the intervention location, for instance, a stenosis region in the vessel, after two bends or curves, a balloon (balloon catheter) or a balloon stent catheter may be pushed by means of an appropriate probe through the working lumen to the intervention location, possibly after removal of the guide wire. It is further possible to take, by means of a push rod or a pushing tool, a stent, in particular an expanding stent, through the working lumen 11 to the intervention location at the distal end of the guide catheter 1. It is further possible to take a micro surgical tool, in particular a drill, into this region so as to perform a therapeutic treatment in the intervention region by means of rotablation, for instance, in the case of a calcified or sidled stenosis. It is also possible to provide one or a plurality of through-holes in an implanted stent by means of the micro drill so as to open accesses to branched-off vessels. Through a lumen 15 that is possibly provided additionally in the guide catheter 1, it is possible to supply a contrast medium or a rinsing solution or the like.

The lengths of the first catheter tube 2 and of the second catheter tube 3 may be dimensioned as a function of the respective medical indication and in particular as a function of the kind and shape of the vessels in which the respective interventions are to be performed. Preferably, a guide catheter 1 with an outer diameter of approximately 5 French or less is used.

### LIST OF REFERENCE SIGNS

1 guide catheter
2 control wire
3 control wire
4 distal catheter end
5 lumen for the control wires
6 lumen for the control wires
7 first catheter tube
8 second catheter tube
9 adapter
10 balloon
11 working lumen
12 slider
13 slider
14 balloon lumen
15 additional lumen
16 guide tube
17 guide tube
18 slider housing
19 slider housing
20-24 locking screws

1. A device for percutaneous vascular intervention, particularly for performing a stent implantation, having a guide catheter and a guide wire that is guided in a catheter lumen of the guide catheter to the intervention location in the vessel, characterized in that two control wires (2, 3) connected with the distal catheter end (4) are guided to be moved in the longitudinal direction in the guide catheter (1), wherein, on actuation of the first control wire (3), the distal catheter end (4) is bent in a first direction and, on actuation of the second control wire (2), is bent in a second direction opposite to the first direction.

2. The device according to claim 1, characterized in that the first control wire (3), with a catheter end (4) bent in the first direction, is adapted to be fixed against a longitudinal displacement.

3. The device according to claim 1, characterized in that the two control wires (2, 3) are guided in longitudinal direction in the catheter cross-section in lumens (5, 6) in the guide catheter (1) which are diametrical to each other.

4. The device according to claim 1, characterized in that the guide catheter (1) is designed to be torsion-free about the longitudinal axis along the entire length thereof.

5. The device according to claim 1, characterized in that each of the two control wires (2, 3) is designed to be torsion-free about the respective longitudinal axis thereof.

6. The device according to claim 1, characterized in that the guide catheter (1) comprises two multi-lumen catheter tubes (7, 8) of different rigidity, a first catheter tube (7) thereof being designed to be relatively rigid, and a second catheter tube (8) that is connected with the distal end of the first catheter tube (7) being designed as a retrogradely bendable tube, and in that the two control wires (2, 3) are connected with the distal end of the second catheter tube (3).

7. The device according to claim 1, characterized in that the proximal end of the first catheter tube (7) is connected in a rotation resistant manner with an adapter (9).

8. The device according to claim 1, characterized in that a balloon (10) is provided at the second catheter tube (8) and in that the guide catheter (1) comprises a balloon lumen (14) via which a filler for filling the balloon (10) may be supplied.

9. The device according to claim 1, characterized in that the guide catheter (1) further comprises a working lumen (11) designed for guiding the guide wire.

10. The device according to claim 1, characterized in that the working lumen (11) is further designed for guiding a micro surgical therapy element, in particular a tube or another therapeutical mechanic element, for instance, a balloon, or for the implantation of a stent.

11. The device according to claim 1, characterized in that the working lumen (11) is designed for the direct injection of contrast medium into the vessel to be intervened.

12. The device according to any of claim 1, characterized in that the guide catheter (1) and the control wires (2, 3) are adapted to be adjusted in different positions of angle of rotation relative to each other about the longitudinal axis of the catheter.

13. The device according to claim 1, characterized in that each control wire (2, 3) is connected at the proximal end thereof with a slider (13).

14. The device according to any of claim 1, characterized in that one of the sliders or both sliders (12, 13) are adapted to be fixed in different positions or by self-locking, respectively.

15. The device according to claim 1, characterized in that it is designed for percutaneous coronary intervention.

16. The device according to claim 1, characterized in that it is designed for a vascular intervention of the arteria carotis.
